(19) United States
(12) Patent Application Publication
(10) Pub. No.: US 2022/0000408 A1
(43) Pub. Date: Jan. 6, 2022

(54) METHODS FOR MITIGATING BIOFOULING EFFECTS OF BIOFLUID INTERFERENTS TO DETECT IN VIVO BIOCHEMICAL AND WEARABLE DEVICE THEREFOR

(71) Applicant: The Regents of the University of California, Oakland, CA (US)

(72) Inventors: Sam EMAMINEJAD, Los Angeles, CA (US); Shuyu LIN, Los Angeles, CA (US)

(73) Assignee: The Regents of the University of California, Oakland, CA (US)

(21) Appl. No.: 17/368,694

(22) Filed: Jul. 6, 2021

Related U.S. Application Data
(60) Provisional application No. 63/048,593, filed on Jul. 6, 2020.

(51) Int. Cl. A61B 5/263; A61B 5/256; A61B 5/00

(52) U.S. Cl. CPC A61B 5/263; A61B 5/256; A61B 5/4277; A61B 2562/247; A61B 2562/125; A61B 2562/0209; A61B 5/4266

(57) ABSTRACT

Example implementations include a device with an electrode electrically responsive to presence of a biochemical present within a biofluid, and one or more biofouling and interferent mitigation layers disposed on the electrode to block transmission of biofouling agents to the electrode and the reaction of interferents on the electrode. Example implementations also include a method of obtaining a biofluid sample, mitigating a biofouling characteristic associated with the biofluid sample, and obtaining a biochemical characteristic associated with the biofluid sample.

### CROSS-REFERENCE TO RELATED PATENT APPLICATIONS

This application claims priority to U.S. Provisional Patent Application Ser. No. 63/048,593, entitled “NONINVASIVE WEARABLE ELECTROACTIVE PHARMACEUTICAL MONITORING FOR PERSONALIZED PHARMACOTHERAPY,” filed Jul. 6, 2020, the contents of such application being hereby incorporated by reference in its entirety and for all purposes as if completely and fully set forth herein.

### STATEMENT REGARDING FEDERALLY SPONSORED RESEARCH OR DEVELOPMENT

This invention was made with government support under Grant Number 1847729, awarded by the National Science Foundation. The government has certain rights in the invention.

### TECHNICAL FIELD

The present implementations relate generally to wearable sensors, and more particularly to mitigating biofouling effects of biofluid interferents to detect an in vivo biochemical.

### BACKGROUND

To realize the vision of personalized medicine, which aims to deliver the right patient, with the right drug, at the right dose, personalized pharmacotherapy solutions are necessary. Currently, medication dosage is generally prescribed by relying on the drug manufacturer's recommendation, which is based on statistical averages obtained from testing the medication on a relatively small patient sample size. Therefore, the recommended dosage may fall outside the optimal therapeutic concentration window, resulting in adverse events in patients and/or ineffective pharmacotherapy. To address such issues, personalized therapeutic drug monitoring (TDM) is essential, as it can guide dosing by capturing the dynamic pharmacokinetic profile of the patient's prescribed medication during the course of the treatment. However, conventional TDM techniques demonstrate invasiveness, high cost, and long turn-around time, due to repeated blood draws and assays performed in offsite central labs.

### SUMMARY

Example implementations include a wearable voltammetric sensor configured to detect acetaminophen (APAP) in biofluids including saliva and blood. APAP is a model electroactive drug molecule and widely-used analgesic and antipyretic. A biochemical sensor in accordance with present implementations can include a biochemical sensor electrode with surface termination adjusted to decouple undesired interference and a polymeric membrane to reject surface-active agents. Surface termination can be adjusted via tuning electron transfer kinetics pertaining to redox reactions. A polymeric membrane can block biofouling interferents to further reject undesired interference with accurate APA detection for in vivo biofluids. Thus, a technological solution for mitigating biofouling effects of biofluid interferents to detect an in vivo biochemical is provided.

Example implementations include a device with an electrode electrically responsive to presence of a biochemical present within a biofluid, and one or more biofouling mitigation layers disposed on the electrode to block transmission of biofouling agents to the electrode and the reaction of interferents on the electrode.

Example implementations also include a device where the electrode includes a boron-doped diamond electrode.

Example implementations also include a device where the biofouling and interferent mitigation layers include an adsorption layer.

Example implementations also include a device where the biofouling and interferent mitigation layers are negatively charged.

Example implementations also include a device where the biofouling and interferent mitigation layers include Nafion.

Example implementations also include a device where the electrode includes at least one of boron-doped diamond, oxygen-terminated boron-doped diamond, and hydrogen-terminated boron-doped diamond.

Example implementations also include a device where the electrode is noninvasively contactable with a biological surface.

Example implementations also include a device where the biological surface includes at least one of skin and human skin.

Example implementations also include a device where the device includes a smartwatch.

Example implementations also include a device with a microfluidic layer disposed on the biofouling and interferent mitigation layers and including at least one microfluidic channel.

Example implementations also include a device where the microfluidic channel includes at least one outlet at a first face of microfluidic layer proximate to the electrode, and at least one inlet at a second face of the microfluidic layer opposite to the first face.

Example implementations also include a device where the microfluidic layer includes at least one flexible layer.

Example implementations also include a method of obtaining a biofluid sample, mitigating an interferent characteristic associated with the biofluid sample; mitigating a biofouling characteristic associated with the biofluid sample, and obtaining a biochemical characteristic associated with the biofluid sample, and extracting one or more biochemical signals from a raw electrochemical readout with a baseline estimation algorithm.

Example implementations also include a method where the biofluid includes at least one of sweat, saliva, human sweat, and human saliva.

Example implementations also include a method where the biofouling and the interferent characteristics are associated with at least one of uric acid, tyrosine, tryptophan, ascorbic acid, histidine, methionine, a protein, a peptide, a lipid, and an amino acid.

Example implementations also include a method where the biochemical characteristic is associated with acetaminophen.

Example implementations also include a method of further contacting an electrode noninvasively with a biological surface, where the obtaining the biofluid sample further includes obtaining the biofluid sample onto the electrode, and where the obtaining the biochemical characteristic further includes obtaining the biochemical characteristic associated with the biofluid sample by the electrode.

Example implementations also include a method of further contacting an electrode including one or more biofouling and interferent mitigation layers noninvasively with a biological surface, where the mitigating the biofouling characteristic further includes mitigating the biofouling characteristic associated with the biofluid sample by the biofouling mitigation layer, wherein the mitigating the interferent characteristic further comprises mitigating the interferent characteristic associated with the biofluid sample by one or more of adjusting surface chemistry of the electrode and incorporating an interferent mitigation layer.

Example implementations also include a method of further repelling surface-active agents and electroactive interferent molecules associated with the biofluid sample, where the surface-active agents comprise the biofouling characteristic, and the electroactive interferent can react on the electrode surface and confound the biochemical signal; the biofouling and interferent mitigation layer repel these molecules from approaching the electrode surface via electrostatic force and size-dependent filtering effect.

Example implementations also include a method of manufacturing an electrode, the electrode being electrically responsive to a biochemical in a biofluid and mitigating a biofouling characteristic associated with an interferent in the biofluid, by applying an anodic treatment to a boron-doped diamond electrode, coating the electrode with a Nafion solution, and drying the Nafion solution to form a mitigation layer on the electrode.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1A illustrates a top view and a bottom view of a first example device in accordance with present implementations.

FIG. 1B illustrates a top view and a bottom view of a second example device in accordance with present implementations.

FIG. 2 illustrates a cross-sectional view of an example biochemical sensor in accordance with present implementations.

FIG. 3A illustrates a first plan view of an example biochemical sensor further to the example cross-sectional view of FIG. 2.

FIG. 3B illustrates a second plan view of an example biochemical sensor further to the example cross-sectional view of FIG. 2.

FIG. 3C illustrates a third plan view of an example biochemical sensor further to the example plan view of FIG. 3B.

FIG. 4 illustrates an example electronic sensor device, in accordance with present implementations.

FIG. 5 illustrates an example biochemical sensor response, in accordance with present implementations.

FIG. 6 illustrates an example biochemical sensor response including a biochemical sensor window, in accordance with present implementations.

FIG. 7 illustrates an example biochemical sensor response corresponding to in vivo biochemical concentration over time, in accordance with present implementations.

FIG. 8 illustrates an example method of electrically sensing a biochemical, in accordance with present implementations.

FIG. 9 illustrates an example method of electrically sensing a biochemical further to the example method of FIG. 8.

FIG. 10 illustrates an example method of manufacturing a device for mitigating biofouling effects of biofluid interferents to detect an in vivo biochemical, in accordance with present implementations.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting; rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

Fundamental challenges inherent to complex biofluid analysis adversely impact conventional systems. One such challenge is the distortion/burial of the target's redox signature in the measured voltammogram, which is due to superimposing voltammetric responses of endogenous electroactive species (“interference”). The characterization of the electroactive interferent species' response leads to the identification of “undistorted potential windows,” within which reliable electroactive target detection in sweat matrix may be demonstrated. To generalize this methodology and apply it to the targets with redox peaks falling outside the original undistorted potential windows, surface engineering strategies are needed to tune the target/interference-surface interactions such that the target redox peaks fall within the undistorted potential windows. Additionally, biofouling is another challenge relevant to the context at hand, which is widely investigated for the conventional biofluids (e.g., blood), but overlooked in the context of sweat analysis. Biofouling stems from the adsorption of surface-active agents (e.g., proteins, peptides, amino acids) onto the sensor's surface. This adsorption layer inhibits the analyte interaction with the electrode, which may lead to signal degradation.

Present implementations include a biochemical sensor device including biofluid channels structures and electrochemical sensors operable to detect target biochemical for in vivo biofluids including sweat and saliva. Example implementations include Nafion-coated and hydrogen-terminated boron-doped diamond electrode (Nafion/H-BDDE), which mitigates biofouling and creates undistorted potential windows corresponding to and substantially encompassing an oxidation peak associated with APAP. Thus, present implementations can demonstrate accurate and reliable quantification of APAP in saliva and sweat. Accordingly, wearable electronic devices in accordance with present implementations can realize real-time and accurate noninvasive biochemical detection for in vivo biofluids within a compact footprint.

Wearable devices in accordance with present implementations can include the biochemical sensor as discussed above within a wearable device, smartwatch, or the like, capable of sweat sampling/routing, signal acquisition, and data display/transmission. The wearable device can further include electronic devices, electrical devices or the like, implementing on-board or substantially on-board processing of voltammetric readouts to detect target redox peak extraction. Such a wearable device is advantageous for clinical, medical, and like use, by providing noninvasive and real-time detection of APAP at least in sweat. By harnessing the demonstrated real-time and reliable drug quantification capabilities, present implementations are advantageously directed to a viable therapeutic drug monitoring approach to enable personalized pharmacotherapy.

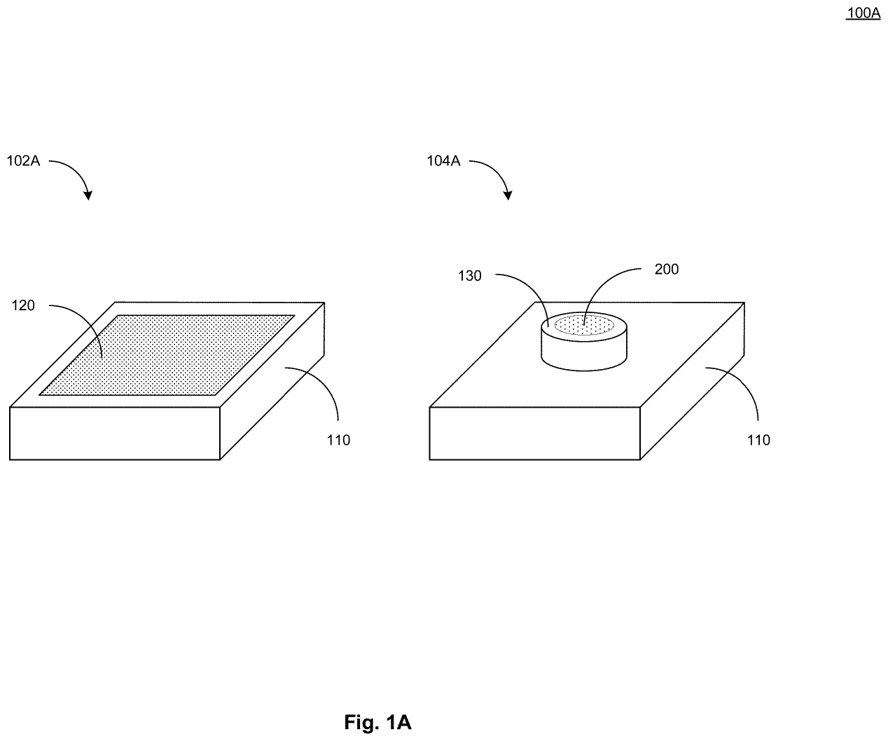

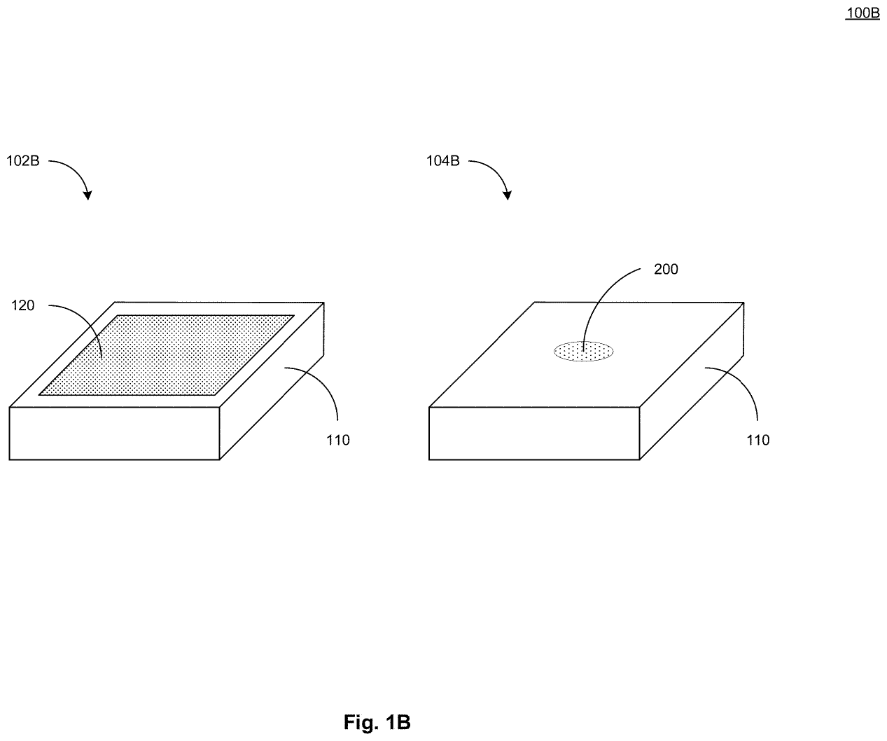

FIG. 1A illustrates a top view and a bottom view of an example device in accordance with present implementations. As illustrated by way of example in a top view 102A and a bottom view 104A of FIG. 1A, the example device 100 includes a housing 110, a display device 120, a stimulation module 130, and an electrochemical sensor 200.

The housing 110 contains or the like one or more sensors, electrical devices, electronic devices, mechanical structures, and the like. In some implementations, the housing 110 includes a plastic material, a polymer material, electrically insulating material, waterproof material, water resistant material, or the like. In some implementations, the housing 110 includes a 3D-printed structure. In some implementations, the housing 110 includes a first face oriented or orientable toward a biological surface. In some implementations, the housing 110 includes a second face oriented or orientable away from the biological surface. In some implementations, the first face and the second face of the housing 110 are disposed on opposite surfaces of the housing 110.

The display device 120 is operable to display one or more biochemical characteristics associated with a biofluid. In some implementations, the biofluid includes one or more characteristics associated with a biochemical therein. In some implementations, the biofluid includes one or more of glucose, choline, and lactate. In some implementations, the characteristics include a pH characteristic. In some implementations, the display device 120 includes an electronic display. In some implementations, the electronic display includes a liquid crystal display (LCD), a light-emitting diode (LED) display, an organic light-emitting diode (OLED) display, or the like. In some implementations, the display device 120 is housed at least partially within the housing 120, on its second face oriented or orientable away from the biological surface.

The stimulation module 130 is operable to apply electrical energy to the biological surface according to one or more electrical output patterns. In some implementations, the stimulation module 130 is operable to apply electrical energy to the biological surface in accordance with an iontophoresis process. In some implementations, the stimulation module 130 is operable to induce a biological reaction from the biological surface. In some implementations, a biological reaction includes release of biofluid from the biological surface. As one example, the stimulation module 130 can apply electrical energy to skin to induce release of sweat. In some implementations, the stimulation module 130 includes one or more electrical, electronic, and logical devices. In some implementations, the stimulation module 130 includes one or more integrated circuits, transistors, transistor arrays, or the like.

The electrochemical sensor 200 is operable to detect one or more biochemicals in contact therewith or contactable therewith. In some implementations, the electrochemical sensor 200 is operable to detect a plurality of biochemical. In some implementations, the electrochemical sensor 200 includes one or more electrode with biochemically-sensitive electrode terminals. In some implementations, the electrochemical sensor 200 includes a plurality of electrodes arranged in a geometric pattern. As one example, the plurality of electrodes can be arranged in a grid pattern including an arbitrary number of electrodes in a length direction and a width direction perpendicular to the length direction. In some implementations, the electrochemical sensor 200 include at least one opening, chamber, or the like, to receive biofluid from the biological surface and to contactably couple the biofluid to at least one electrode terminal, biochemically-sensitive electrode terminal, or a combination thereof. In some implementations, the biochemical sensor 140 includes one or more polymers, plastics, or the like. In some implementations, the biochemical sensor 140 includes one or more films, sheets, layers, or the like. In some implementations, the biochemical sensor 140 is or includes one or more films, sheets, layers, or the like arranged in a planar structure. In some implementations, the biochemical sensor 140 is or includes a flexible structure deformable, bendable, or the like in one or more planar directions.

FIG. 1B illustrates a top view and a bottom view of a second example device in accordance with present implementations. As illustrated by way of example in a top view 102B and a bottom view 104B of FIG. 1B, the example device 100B includes the housing 110, the display device 120, and the electrochemical sensor 200. In some implementations, the example device 100B includes the housing 110 and the display device 120 correspondingly to those of the example device 100A. In some implementations, the example device 100B does not include the stimulation module 130, and the electrochemical sensor 200 is disposed on, over, in, or the like, the housing 110.

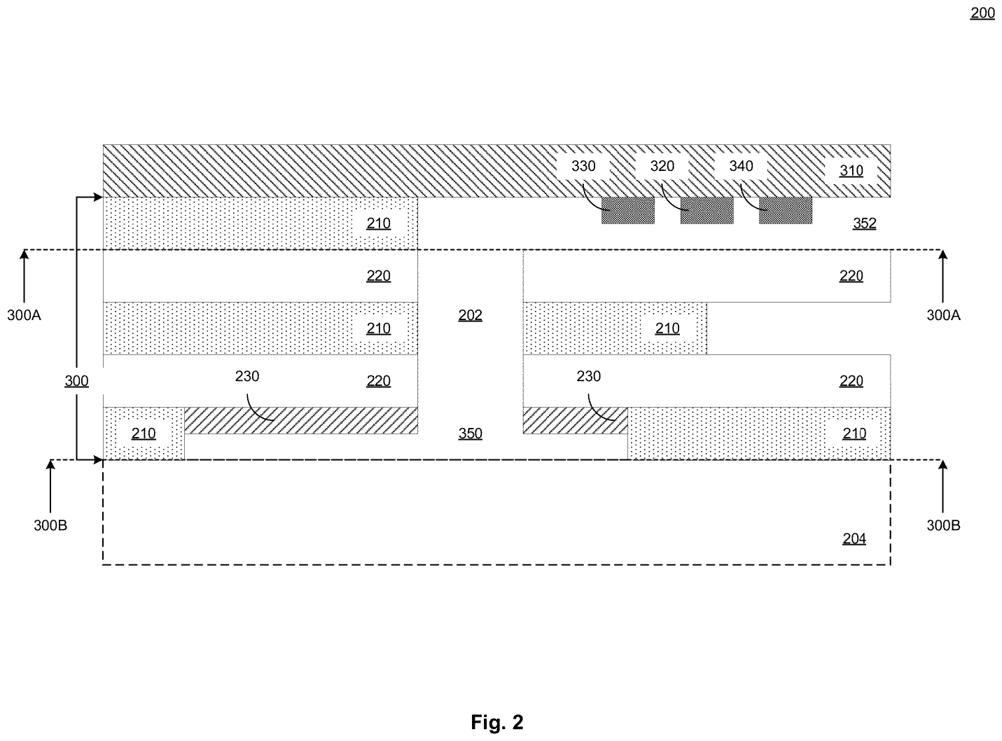

FIG. 2 illustrates a cross-sectional view of an example biochemical sensor in accordance with present implementations. As illustrated by way of example in FIG. 2, an example biochemical sensor 200 includes a sensor substrate 310, a biochemical sensor electrode 320, a counter electrode 330, a reference electrode 340, and a microfluidic layer 300 including one or more of adhesive layers 210, polymer layers 220, a spacer layer 230, a biofluid inlet 350, a biofluid transportation channel 202, and a biofluid outlet 352. The biochemical sensor 200 can be contactable with a biological surface of a biological object 204. The sensor substrate 310 can include a substantially planar structure on which one or more electrical sensors, electrochemical sensors, or the like, are disposed, patterned, affixed, or the like. The sensor substrate 310 can include a flexible substrate, a flexible solid material, or the like.

The adhesive layers 210 are stackably disposed in a direction substantially orthogonal to a plane of the adhesive layers 210. The adhesive layers 210 can be bonded, affixed, attached, or the like, to each other, a biological substrate of the biological object 204, or the sensor substrate 310. The adhesive layers 210 can also be bonded, affixed, attached, or the like, to the polymer layers 220. In some implementations, the adhesive layers can be stackably disposed alternatingly with the polymer layers 220. One or more of the adhesive layers 210 can include a flexible planar substrate and an adhesive coating on one or more planar surfaces thereof. As one example, the adhesive layers 210 can include double-sided adhesive tape having a thickness of approximately 170 μm.

The polymer layers 220 are stackably disposed in a direction substantially orthogonal to a plane of the polymer layers 220. The polymer layers 220 can be bonded, affixed, attached, or the like, to one or more of the adhesive layers 210. One or more of the polymer layers 220 can include a flexible planar substrate. As one example, the polymer layers 220 can include polyethylene terephthalate (PET) sheets having a thickness of approximately 170 μm. The spacer layer 230 is stackably disposed in a direction substantially orthogonal to a plane of the spacer layer 230. The spacer layer 230 can be bonded, affixed, attached, or the like, to one or more of the adhesive layers 210. The spacer layer 230 can provide fine control over the volume of the biofluid inlet, allowing biofluid to be transported through the chamber without pooling at the biofluid inlet 350. One or more of the spacer layer 230 can include a flexible planar substrate. As one example, the spacer layer 230 can include one or more stacked layers of single-sided laminating sheets.

The biofluid inlet 350 includes at least one opening, cavity or the like in the microfluidic layer 300 disposed away from the sensor substrate 310. The biofluid inlet 350 can be placed proximate to the biological surface of the biological object 204, and can be adhesively contacted to the biological surface to form a substantially watertight seal therewith. The biofluid transportation channel 202 includes at least one opening, cavity or the like in the microfluidic layer 300 proximate to the sensor substrate 310. The biofluid transportation channel 202 can be placed proximate to the biological surface of the biological object 204, and can couple the biofluid inlet 350 to the biofluid outlet 352. The biofluid transportation channel 202 can thus transport biofluid from the biological surface of the biological object 204 vertically through the microfluidic layer 300 toward the sensor substrate 310 and the electrodes 320, 330 and 340 for sensing of at least one biochemical therein. The biofluid outlet 352 includes at least one opening, cavity or the like in the microfluidic layer 300 disposed proximate to the sensor substrate 310. The biofluid outlet 352 can be placed proximate to the electrodes 320, 330 and 340 of the sensor substrate 310, and can expel biofluid out of the device 100 to the biological surface after passing the biofluid to the electrodes 320, 330 and 340 for sending of the biochemical therein.

The biological object 204 includes the biological surface and is or includes living tissue, biological matter, or the like. In some implementations, the biological object 204 is or includes human skin, animal skin, and the like. In some implementations, the biological object 204 includes, directs or is responsive to secrete one or more biofluids. As one example, the biological object 204 can be responsive to electrical stimulation to induce secretion of sweat by an iontophoresis process or the like.

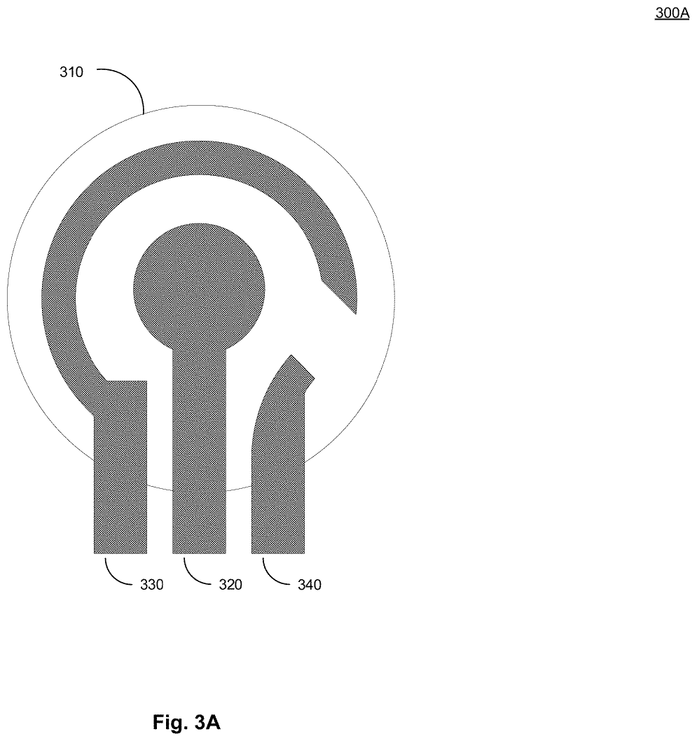

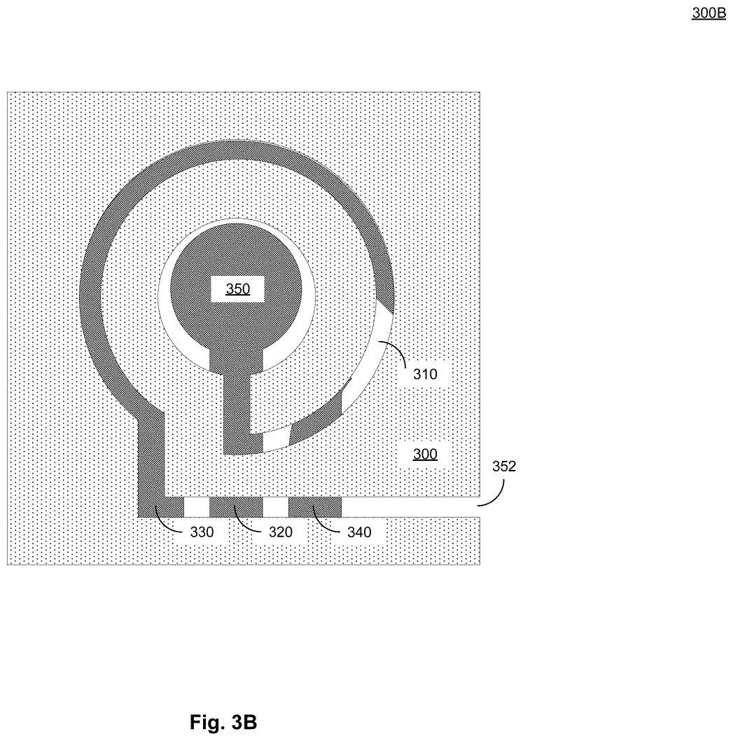

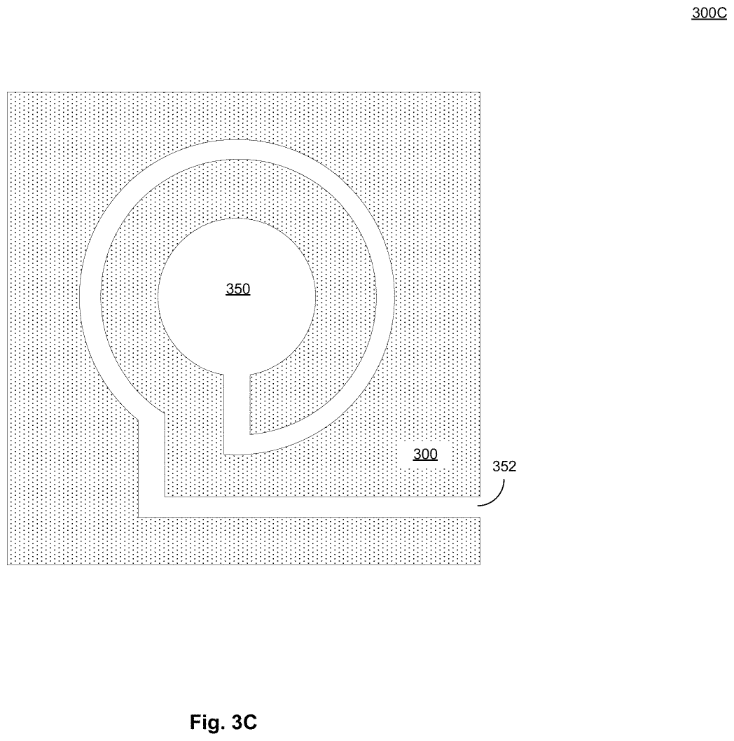

FIG. 3A illustrates a first plan view of an example biochemical sensor further to the example cross-sectional view of FIG. 2. As illustrated by way of example in FIG. 3A, an example first plan view 300A includes the sensor substrate 310, the biochemical sensor electrode 320, the counter electrode 330, and the reference electrode 340.

The biochemical sensor electrode 320 is operable to receive a response current responsive to the presence of a biochemical. In some implementations, the biochemical sensor electrode 320 is a boron-doped diamond electrode (BDDE). BDDE possess advantageous properties including but not limited to a wide electrochemical potential window and high operational stability. A surface of the biochemical sensor electrode contactable with a biological surface can be treated with a coating, additive, or the like. In some implementations, a biochemical sensor electrode 320 is operable to detect the presence of one or more biochemicals in a biofluid at nanomolar and micromolar levels. Because of its unique sp3 diamond structure, BDDE advantageously manifests various electrochemical sensing properties including a wide electrochemical potential window, low background current, high fouling resistance, high biocompatibility, relatively rapid electron transfer kinetics, and long term stability under high-potential operation. In some implementations, the biochemical sensor electrode 320 is an anodic-treated BDDE. BDDE can manifest a double layer capacitance of substantially 8 μF/cm2, indicating a low background current when applied in voltammetric measurements.

The counter electrode 330 is optionally integrated into the example biochemical sensor 200. In some implementations, the counter electrode is a glassy carbon electrode (GCE). In some implementations, the counter electrode 220 is a screen printed carbon electrode. The reference electrode 340 is operable to apply one or more current pulses to a biological surface. In some implementations, the reference electrode 230 is or includes silver.

FIG. 3B illustrates a second plan view of an example biochemical sensor further to the example cross-sectional view of FIG. 2. As illustrated by way of example in FIG. 3B, an example second plan view 300B includes the microfluidic layer 300, the sensor substrate 310, the biochemical sensor electrode 320, the counter electrode 330, the reference electrode 340, the biofluid inlet 350, and the biofluid outlet 352.

FIG. 3C illustrates a third plan view of an example biochemical sensor further to the example plan view of FIG. 3B. As illustrated by way of example in FIG. 3C, an example second plan view 300B includes the microfluidic layer 300, the biofluid inlet 350, and the biofluid outlet 352, and omits the sensor substrate 310, the biochemical sensor electrode 320, the counter electrode 330, and the reference electrode 340 for illustrative purposes.

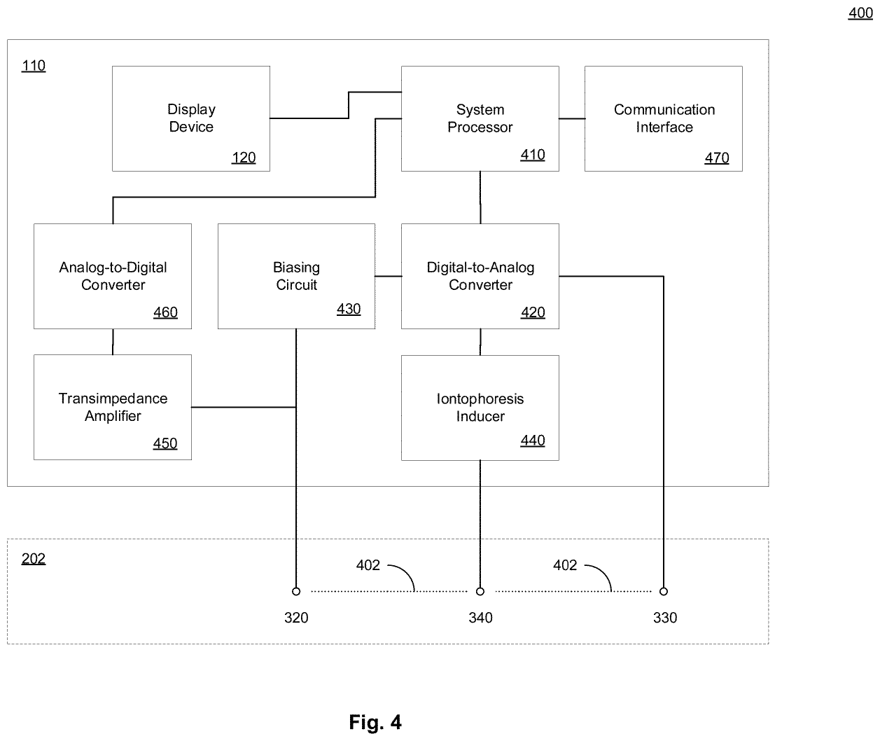

FIG. 4 illustrates an example electronic sensor device, in accordance with present implementations. As illustrated by way of example in FIG. 4, example electronic sensor device 400 includes the sensor device housing 110 and the display device 120. The electronics region 130 can include the biochemical sensor electrode 320, the counter electrode 330, the reference electrode 340, a system processor 410, a digital-to-analog converter (DAC) 420, a biasing circuit 430, an iontophoresis inducer 440, a transimpedance amplifier (TIA) 450, an analog-to-digital converter (ADC) 460, a communication interface 370. In some implementations, the example electronic sensor device 400 includes and is contactable with a biological surface of the biological object 204 by one or more of the biochemical sensor electrode 320, the counter electrode 330, and the reference electrode 340. In some implementations, the example electronic sensor device 400 interfaces with the biological surface by at least one biological conductive path 402 at the biological surface of the biological object 204. In some implementations, the conductive path 402 is disposed through one or more of the biochemical sensor electrode 320, the counter electrode 330, and the reference electrode 340.

The system processor 410 is operable to execute one or more instructions associated with input from at least one of the biochemical sensor surface 110 and the biochemical sensor electrode 320. In some implementations, the system processor 410 is an electronic processor, an integrated circuit, or the like including one or more of digital logic, analog logic, digital sensors, analog sensors, communication buses, volatile memory, nonvolatile memory, and the like. In some implementations, the system processor 410 includes but is not limited to, at least one microcontroller unit (MCU), microprocessor unit (MPU), central processing unit (CPU), graphics processing unit (GPU), physics processing unit (PPU), embedded controller (EC), or the like. In some implementations, the system processor 410 includes a memory operable to store or storing one or more instructions for operating components of the system processor 410 and operating components operably coupled to the system processor 410. In some implementations, the one or more instructions include at least one of firmware, software, hardware, operating systems, embedded operating systems, and the like. It is to be understood that the system processor 410 or the device 300 generally can include at least one communication bus controller to effect communication between the system processor 410 and the other elements of the device 300. In some implementations, the system processor 410 is operable to generate one or more square wave voltage pulse signal instructions to apply one or more stimulation current pulses to a biological surface. In some implementations, the system processor 410 is operable to apply the current pulses to the reference electrode directly. Alternatively, in some implementations, the system processor is operable to apply the current pulses indirectly by at least one intervening structure. In some implementations, the intervening structure is or includes the DAC 420.

The DAC 420 is operable to receive one or more digital instructions from the system processor 410 and to output one or more analog signals corresponding to the digital instructions. In some implementations, the DAC 420 is operatively coupled to the iontophoresis inducer 440 by at least one communication line, bus, or the like. In some implementations, the DAC 420 supplies one or more analog instructions to the iontophoresis inducer 440 to apply at least one current pulse, sequence of current pulses, and the like, to the reference electrode. In some implementations, the DAC 420 includes one or more logical or electronic devices including but not limited to integrated circuits, logic gates, flip flops, gate arrays, programmable gate arrays, and the like. It is to be understood that any electrical, electronic, or like devices, or components associated with the DAC 420 can also be associated with, integrated with, integrable with, replaced by, supplemented by, complemented by, or the like, the system processor 410 or any component thereof.

The biasing circuit 430 is operable to receive one or more instructions from the DAC 420 to apply an electrical bias to the biochemical sensor electrode 410. In some implementations, the biasing circuit 430 applies a constant voltage at a minimum bias voltage to the biochemical sensor electrode 410. As one example, a minimum bias voltage is equal to an activation voltage of a BDDE. In some implementations, the biasing circuit 430 includes one or more logical or electronic devices including but not limited to integrated circuits, logic gates, flip flops, gate arrays, programmable gate arrays, and the like. It is to be understood that any electrical, electronic, or like devices, or components associated with the biasing circuit 430 can also be associated with, integrated with, integrable with, replaced by, supplemented by, complemented by, or the like, the system processor 410 or any component thereof.

The iontophoresis inducer 440 is operable to control, generate, define, or the like, one or more signals, pulses, or the like, of electrical energy applied to the biological surface according to one or more electrical output patterns. In some implementations, the iontophoresis inducer 440 is operable to apply electrical energy to the biological surface in accordance with an iontophoresis process. In some implementations, the electronics portion 130 of the housing 110 includes the iontophoresis inducer 440. In some implementations, the iontophoresis inducer 440 is operable to induce a biological reaction from the biological surface in accordance with the operation of the reference electrode 340. In some implementations, the iontophoresis inducer 440 includes one or more electrical, electronic, and logical devices. In some implementations, the iontophoresis inducer 440 includes one or more integrated circuits, transistors, transistor arrays, or the like. The reference electrode 340 is operable to apply one or more signals, pulses, or the like, of electrical energy to the biological surface according to one or more electrical output patterns in response to signals, instructions, or the like received from at least one of the DAC 430 and the iontophoresis inducer 440.

In some implementations, the iontophoresis inducer 440 applies a constant voltage at a minimum stimulation voltage to the reference electrode 340. As one example, a minimum stimulation voltage is equal to a lowest voltage magnitude associated with a particular voltage window. In some implementations, the iontophoresis inducer 440 is operable to increase a stimulation voltage in accordance with a step voltage or the like. In some implementations, the iontophoresis inducer 440 is operable to increase a stimulation voltage from the minimum stimulation voltage to a maximum stimulation voltage according to the step voltage, a timing parameter, and the like. As one example, a maximum stimulation voltage is equal to a highest voltage magnitude associated with a particular voltage window. In some implementations, the iontophoresis inducer 440 is operable to apply one or more current pulses to increase or decrease the magnitude of the stimulation voltage applied by the iontophoresis inducer 440.

The TIA 450 is operable to receive a response current from the biochemical sensor electrode 320. In some implementations, the biochemical sensor electrode 320 is operable to transmit a response current of varying magnitudes proportional to of one or more biochemicals present in contact therewith. In some implementations, the TIA 450 receives one or more electrical impulses at one or more current response levels, and converts the current response to a voltage response. In some implementations, the TIA 450 converts the current response to a voltage response based on an actual or estimated resistance, impedance, or like of at least one of the biological surface 380 and the biological conductive path 402. In some implementations, the TUA 450 is operable to temporarily store one or more current responses and voltage responses, at a memory device integrable, couplable, or integrated therewith, or operably coupled thereto. In some implementations, the memory device is or includes an electrically erasable programmable read-only memory (EEPROM). In some implementations, the TIA 450 includes one or more logical or electronic devices including but not limited to integrated circuits, logic gates, flip flops, gate arrays, programmable gate arrays, and the like. It is to be understood that any electrical, electronic, or like devices, or components associated with the TIA 450 can also be associated with, integrated with, integrable with, replaced by, supplemented by, complemented by, or the like, the system processor 410 or any component thereof.

The ADC 460 is operable to receive one or more digital instructions from the TIA 450 and to output one or more analog signals corresponding to the digital instructions to the system processor 410. In some implementations, the ADC 460 is operatively coupled to the system processor 410 and the TIA 450 by at least one communication line, bus, or the like. In some implementations, the ADC 460 receives one or more analog instructions from the TIA 450 including at least one voltage response based on at least one current pulse, sequence of current pulses, and the like, from the biochemical sensor electrode 320. In some implementations, the ADC 460 includes one or more logical or electronic devices including but not limited to integrated circuits, logic gates, flip flops, gate arrays, programmable gate arrays, and the like. In some implementations, the ADC includes a 24-bit address space. It is to be understood that any electrical, electronic, or like devices, or components associated with the ADC 460 can also be associated with, integrated with, integrable with, replaced by, supplemented by, complemented by, or the like, the system processor 410 or any component thereof.

The communication interface 470 is operable to communicatively couple at least the system processor 410 to at least one external device. In some implementations, the communication interface 470 includes one or more wired interface devices, channels, and the like. In some implementations, the communication interface 470 includes, is operably coupled to, or is operably couplable to an I2C, UART, or like communication interface by one or more external devices, systems, or the like. In some implementations, the communication interface 470 includes a network or an Internet communication interface or is operably couplable to an Internet communication interface by one or more external devices, systems, or the like. In some implementations, the communication interface 470 is or includes a wireless transceiver operable to wirelessly and bilaterally communicate user commands and the sensor output current. In some implementations, the communication interface 470 is or includes a Bluetooth™ transceiver. In some implementations, the communication interface 470 communication in real-time with an external device. In some implementations, an external device includes a custom-developed computer, smartphone, tablet, or like application compatible with the output of the system processor 410.

The biological surface of the biological object 204 is or includes a surface of living tissue, biological matter, or the like. In some implementations, the biological surface includes partially or fully exposed skin or the like of a human, animal, plant, or the like. In some implementations, the biological surface secretes or is capable of secreting one or more fluids having one or more biochemicals therein. In some implementations, biochemicals include, but are not limited to, dipyridamole, acetaminophen, caffeine, and the like. In some implementations, a biological surface is a wrist, forearm, or the like.

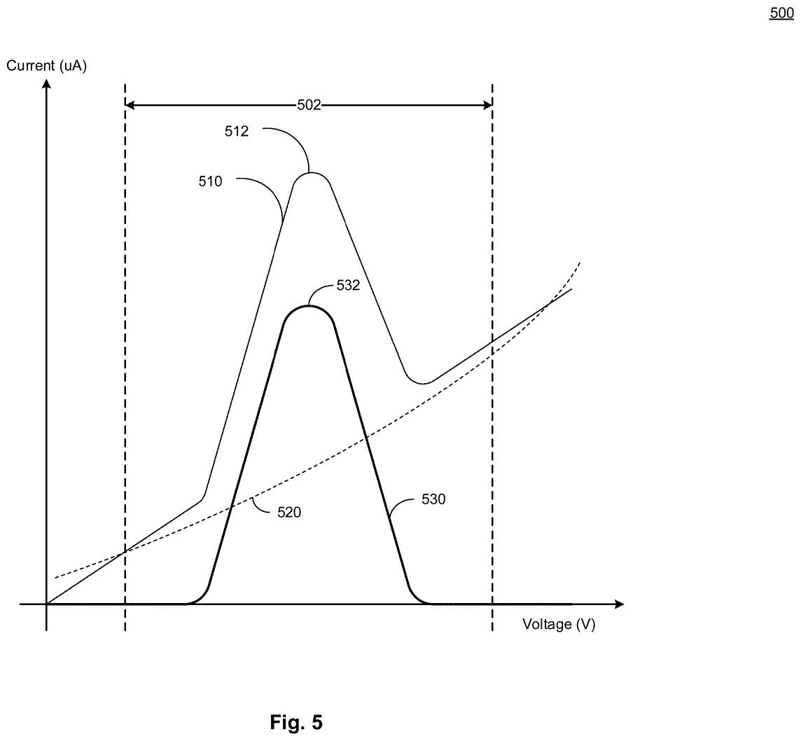

FIG. 5 illustrates an example biochemical sensor response, in accordance with present implementations. As illustrated by way of example in FIG. 5, the example biochemical sensor response is bounded by a characteristic voltage window 502, and includes a characteristic response current curve 510, a characteristic current peak 512, a baseline calibration curve 520, and corrected characteristic response current 530, and a corrected current peak 532. In some implementations, voltammetry-based approaches uniquely leverage the electroactive nature of target drug molecules for quantification, thus eliminating reliance on the availability of recognition elements, mediators, and the like. In some implementations, pulse voltammetry, including but not limited to differential pulse voltammetry (DPV) and square wave voltammetry (SWV) are advantageous for the quantification of electroactive species due to their ability to suppress non-Faradaic background current. In some implementations, an example system sweeps voltage across the biochemical sensor electrode 210 and the reference electrode 230 above redox potential of target electroactive species. As one example, a redox potential is an oxidation potential. In some implementations, a characteristic current peak 512 is recorded at a fingerprint redox voltage associated with a target biochemical, with a peak height correlated to a concentration level of the target biochemical.

The characteristic voltage window 502 includes and bounds a range of voltages associated with the characteristic current peak 512 of the characteristic response current curve 510. In some implementations, the characteristic voltage window 502 includes a predetermined maximum window voltage and a predetermined minimum window voltage. In some implementations, the maximum window voltage and the minimum window voltage of the characteristic voltage window 502 are predetermined to enclose, encompass, bound, or the like, a voltammogram defining an electrochemical response of a biochemical.

The characteristic response current curve 510 defines an electrochemical response of a particular biochemical, and includes a characteristic current peak 512 defining a maximum electrochemical response to voltage stimulation by the iontophoresis inducer 440. In some implementations, the characteristic response current curve 510 is associated with a particular biochemical. In some implementations, the characteristic response current curve 510 includes an electrochemical response from at least one of a target biochemical, background electrochemical activity, and an interferent present with the target biochemical. In some implementations, the characteristic current peak 512 of the characteristic response current curve 510 has a particular current magnitude associated with a concentration of the target biochemical. Thus, in some implementations, the example device 100 determines a concentration of target biochemical present in the biofluid of the biological surface based on a magnitude of a current peak. However, in some implementations, the characteristic current peak 512 is distorted by the presence of background electrochemical activity and an interferent present with the target biochemical. Thus, in some implementations, mitigation of one or more of these distortion drivers is conducted.

The baseline calibration curve 520 defines a level of background electrochemical activity present in the characteristic voltage window 502. In some implementations, the baseline calibration curve 520 is generated based on a curve fitted to a physically detected calibration current response, or a predetermined value based on an estimate thereon. In some implementations, baseline calibration curve 520 is or is based on a combination of a 3rd-order polynomial and exponential equation. The polynomial and exponential equation can include various constants associated with background electrical activity present within the characteristic voltage window 502. Equation 1 (Eq. 1) can correspond to a baseline calibration curve in accordance with present implementations.

I_{baseline}=a₁V³+a₂V+a₃+a₄×e^{a}^{₅}^{V} Eq. 1

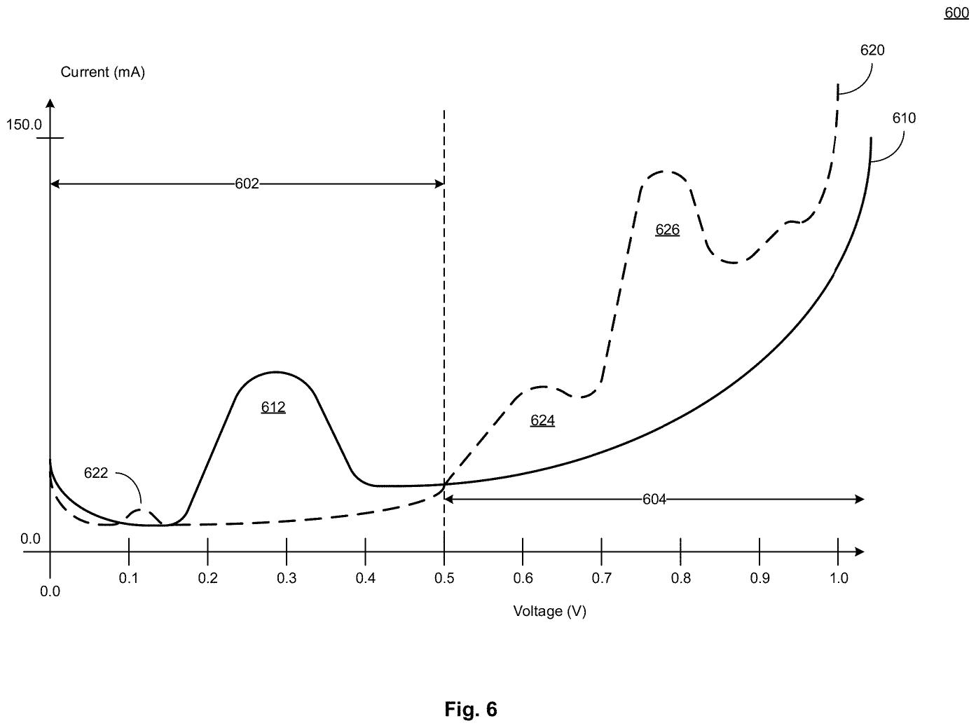

FIG. 6 illustrates an example biochemical sensor response including a biochemical sensor window, in accordance with present implementations. As illustrated by way of example in FIG. 6, an example biochemical sensor response 600 includes a biochemical response curve 610 having a biochemical response peak 612, and a biofouling interferent response curve 620 having a first interference response peak 622 within an undistorted response window 602, and having second and third response peaks 624 and 626 within a interference response window 604.

In some implementations, pulse voltammetry, including but not limited to differential pulse voltammetry (DPV) and square wave voltammetry (SWV) are advantageous for the quantification of electroactive species due to their ability to suppress non-Faradaic background current. To reliably measure the APAP's voltammetric response in biofluids with complex matrices, its redox peak falls within undistorted response window 602, which can include a voltage potential range within which the voltammetric contributions of interfering species are negligible as compared to the voltammetric contribution of APAP. Biofluids, including but not limited to sweat and saliva, can include interferents including but not limited to uric acid (UA), tyrosine (TYR), and tryptophan (TRY) that can be major endogenous contributors to the measured biochemical response curve 610. Additional interferents can include ascorbic acid, histidine, and methionine, proteins, peptides, lipids, and amino acids. Thus, it is advantageous to minimize the effect of interferents within the undistorted response window to realize accurate detection of APA in biofluid. The distorted response window 604 can include a voltage potential range within which the voltammetric contributions of interfering species are significant enough in magnitude to overwhelm the voltammetric response of APAP.

The biochemical response curve 610 defines an electrochemical response of a biochemical in a biofluid, and includes a biochemical response peak 612 defining a maximum electrochemical response to presence of the biochemical in the biofluid. The biochemical response curve 610 can be associated with a particular biochemical, including but not limited to APAP. In some implementations, the biochemical response curve 610 includes an electrochemical response from at least one of a target biochemical, background electrochemical activity, and an interferent present with the target biochemical. In some implementations, the biochemical response peak 612 of the biochemical response curve 610 has a particular current magnitude associated with a concentration of the target biochemical. As one example, the example device 100 can determine a concentration of APAP present in the biofluid of the biological surface based on a magnitude of the biochemical response peak 612. However, in some implementations, the biochemical response peak 612 is distorted by the presence of background electrochemical activity and an interferent present with the target biochemical. Thus, mitigation of one or more of these distortion drivers can be conducted in accordance with the discussion of FIG. 5.

The biofouling interferent response curve 620 defines an electrochemical response of an interferent in a biofluid, and includes first, second, and third interferent response peaks 622, 624 and 626 defining a maximum electrochemical response to presence of one or more interferents in the biofluid. The biofouling interferent response curve 620 can be associated with a particular biochemical, including but not limited to UA, TYR and TRY. In some implementations, the biofouling interferent response curve 620 includes an electrochemical response from at least one of a target biochemical, background electrochemical activity, and an interferent present with the target biochemical. The first interferent response peak 622 of the biofouling interferent response curve 620, within the undistorted response window 602, has a particular current magnitude associated with a concentration of one or more interferents generated away from or below biochemical response peak 612 and causing minimal to no interference with the biochemical response peak 612. The second and third interferent response peaks 624 and 626 are generated at or above the biochemical response curve 610, and introduce significant interference with respect to detection of the target biochemical. Thus, the biochemical response curve 610 can be buried, undetectable, or the like, by the presence of biofouling interferent response curve 620 within an interference response window 604.

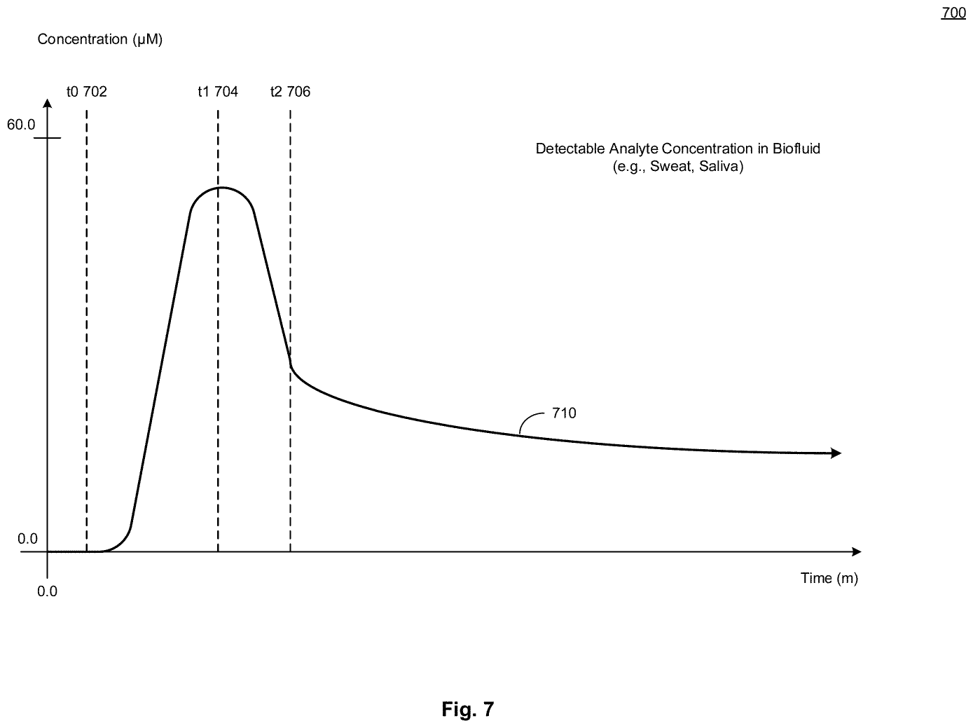

FIG. 7 illustrates an example biochemical sensor response corresponding to in vivo biochemical concentration over time, in accordance with present implementations. As illustrated by way of example in FIG. 7, an example biochemical sensor response 700 includes a time-varying biochemical response curve 710 having characteristic points at time t0 702, time t1 704, and time t2 706. The biochemical response curve 710 can be responsive to detection of APAP secreted, emitted, or the like, from a biological surface of the biological organism, including but not limited to sweat, saliva, and the like. The biochemical response curve 710 can correspond to a particular curve tracking experimentally-validated time-series concentration. As one example, the biochemical response curve 710 can be curve-fitted to Equation 2 (Eq. 2) according to a single-compartment model:

c=A[e−Kₑₗ(t−t₀)−e−Kₐ(t−tₒ)] Eq. (2)

In example Equation 2, t is time after the oral administration of the APAP, t0 is the lag time with respect to the administration time, A is the pre-exponential factor, and Kₑₗ and Kₐ are respectively the elimination and absorption rate constants. The administration time can be effectively the total lag time of oral administration to blood diffusion and blood to sweat or saliva diffusion. Pharmacokinetic parameters and biochemical responses for saliva are similar to each other. Moreover, the resemblance of the fitted pharmacokinetic profiles of sweat and saliva can be similar. Given the readily established saliva-blood correlation of APAP, present implementations support clinical utility of sweat for non-invasive therapeutic drug monitoring.

At time t0 402, the biochemical response curve 710 is responsive to the presence of no or substantially no APAP at a time before ingestion, circulation, or the like of APAP in a biological organism, a person, or the like. As one example, time t0 402 can be associated with a time of +0 minutes, corresponding to a time of ingestion of APAP for a person not having any baseline APAP in their bloodstream, sweat, saliva, or the like. As another example, the amount of APAP can be 650 mg. At time t1 404, the biochemical response curve 710 is responsive to the presence of a substantially peak amount of APAP at a first time after ingestion, circulation, or the like of APAP in a biological organism, a person, or the like. As one example, time t1 404 can be associated with a time of +30 ±15 minutes, corresponding to a time after ingestion of APAP in the bloodstream, sweat, saliva, or the like. At time t2 406, the biochemical response curve 710 is responsive to the presence of a decreasing amount of APAP at a second time after ingestion, circulation, or the like of APAP in a biological organism, a person, or the like. As one example, time t2 406 can be associated with a time of +140±15 minutes, corresponding to a time after ingestion of APAP in the bloodstream, sweat, saliva, or the like. The biochemical response curve 710 can continue to decrease after time t2 406.

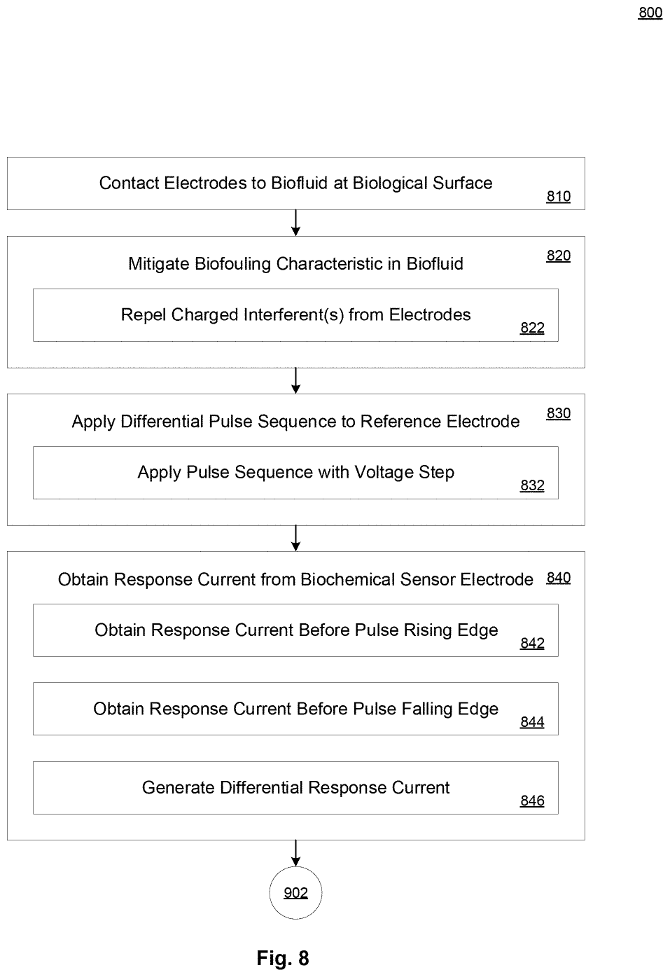

FIG. 8 illustrates an example method of electrically sensing a biochemical, in accordance with present implementations. In some implementations, the example device 100 performs method 800 according to present implementations. In some implementations, the method 800 begins at step 810.

At step 810, the example system contacts electrodes to a biological surface. In some implementations, the biochemical sensor device 100 is a wearable device attached, affixed, or the like to a biological surface of an individual user's body. In some implementations, the biochemical sensor device is attached to a limb, arm, forearm, hand, or the like. In some implementations, the biochemical sensor surface 200 is disposed in contact with the biological surface 202, such that one or more of the biochemical sensor electrode 320, the counter electrode 330, and the reference electrode 340 are in contact with the biological surface 202. The method 800 then continues to step 820.

At step 820, the example system mitigates a biofouling characteristic in the biofluid. The example system can mitigate biofouling characteristics by substantially reducing or preventing contact by one or more interferents with one or more electrodes 320, 330 and 340, while concurrently permitting contact by at least one target biochemical with one or more of the electrodes 320, 330 and 340. In some implementations, step 820 includes step 822. At step 822, the example system repels one or more charged interferents from one or more of the electrodes. The method 800 then continues to step 830.

At step 830, the example system applies a differential pulse sequence to a reference electrode. In some implementations, system processor 410 determines one or more parameters governing the electrical characteristics of the differential pulse sequence. In some implementations, the system processor 410 determines at least one of a pulse amplitude, a pulse period between pulses, a pulse width of each pulse, and a step magnitude of the differential pulse sequence. In some implementations, a pulse amplitude is between 0.0 V and 0.2 V. In some implementations, a pulse period is greater than 0.5 s. In some implementations, a pulse width is less than 0.2 s. In some implementations, step 830 includes step 832. At step 832, the example system applies a pulse sequence with a voltage step. The voltage step can be monotonically increasing. The voltage step can cause the differential pulse sequence to monotonically increase from a minimum voltage associated with a voltage window to a maximum voltage associated with the voltage window. The voltage step can be added to a falling edge of the pulse. Thus, in some implementations, the voltage pulse ends at an ending voltage after the pulse that is higher than a starting voltage before the pulse, by an amount of the voltage step. The method 800 then continues to step 850.

At step 840, the example system obtains a response current from a biochemical sensor electrode. In some implementations, the system processor 410 obtains the response current from one or more of the TIA 450 and the ADC 460. In some implementations, the example system obtains the response current as an analog signal responsive to physical biochemical input, and generates a digital instruction, value, or the like, based on the analog signal. In some implementations, step 840 includes at least one of steps 842, 844 and 846. At step 842, the example system obtains a response current before a pulse rising edge. In some implementations, at least one of the system processor 410, the TIA 450 and the ADC 460 detects and captures a rising edge sample prior to the occurrence of a rising edge pulse. At step 844, the example system obtains a response current after a pulse falling edge. In some implementations, at least one of the system processor 410, the TIA 450 and the ADC 460 detects and captures a rising edge sample after the occurrence of a falling edge pulse. At step 846, the example system generates a differential response current. In some implementations, the system processor 410 generates the differential response current by averaging or the like two adjacent rising edge samples. In some implementations, the system processor 410 generates the differential response current by averaging or the like two adjacent falling edge samples. In some implementations, the method 800 then continues to step 902.

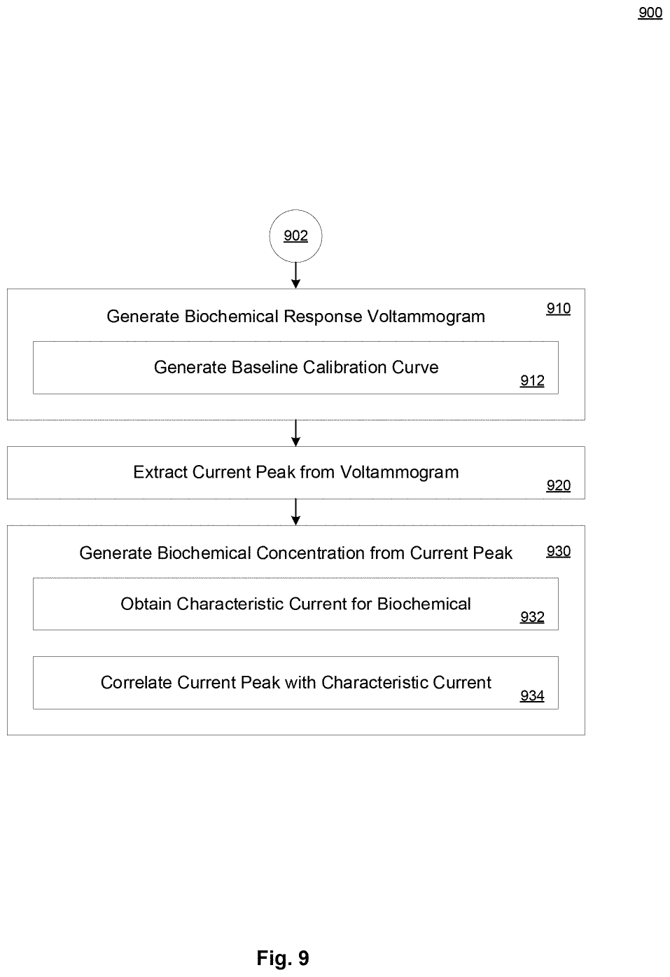

FIG. 9 illustrates an example method of electrically sensing a biochemical further to the example method of FIG. 8. In some implementations, the example device 100 performs method 900 according to present implementations. In some implementations, the method 900 begins at step 802. The method 900 then continues to step 810.

At step 910, the example system generates a biochemical response voltammogram. In some implementations, the system processor 410 generates the biochemical response voltammogram by obtaining voltage and current response pairs. In some implementations, the biochemical response voltammogram includes a plurality of voltage and current response pairs respectively based on the differential response currents and the voltage magnitudes monotonically creasing by voltage step. In some implementations, step 910 includes step 912. At step 912, the example system generates a baseline calibration curve. In some implementations, the system processor 410 generates, obtains, or the like, the baseline calibration curve in accordance with Eq. 1. The method 900 then continues to step 920.

At step 920, the example system extracts a current peak from the voltammogram. In some implementations, the system processor 410 transmits the voltammogram to an external processor, remote device, or the like, by the communication interface 470. In some implementations, the example system extracts a current peak for APAP in the presence of one or more of UA, TRY, TYR, HIS and MET. The method 900 then continues to step 930.

At step 930, the example system generates a biochemical concentration from the current peak. In some implementations, the system processor 410 generates the biochemical concentration. In some implementations, step 930 includes at least one of steps 932 and 934. At step 932, the example system obtains a characteristic current for a biochemical. In some implementations, the system processor obtains, generates, or the like, a predetermined relationship between response current and a concentration associated with a particular biochemical. As one example, the system processor 410 can retrieve a correlation between biochemical concentrations and current response magnitudes for APAP. In some implementations, the correlation defines one or more linear or nonlinear relationships between response current magnitude and concentration of a particular biochemical. At step 934, the example system correlates the current peak with the characteristic current. In some implementations, the system processor 410 generates the biochemical concentration based on the magnitude of the extracted peak with respect to a linear, nonlinear, or like function correlating a biochemical with a ranges of concentrations based on magnitude of response current. In some implementations, the method 900 ends at step 930.

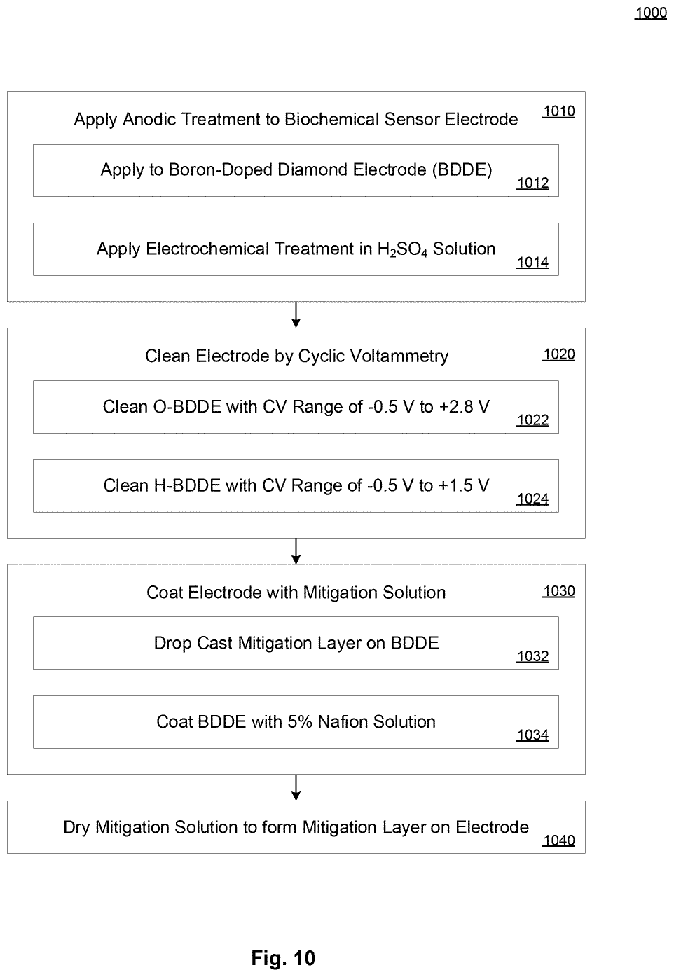

FIG. 10 illustrates an example method of manufacturing a device for mitigating biofouling effects of biofluid interferents to detect an in vivo biochemical, in accordance with present implementations. In some implementations, at least one of the example device 100 is manufactured by method 1000 according to present implementations. In some implementations, the method 1000 begins at step 1010.

At step 1010, the example system applies an anodic treatment to a biochemical sensor electrode. In some implementations, step 1010 includes at least one of steps 1012 and 1014. At step 1012, the example system applies an anodic treatment to a boron-doped diamond electrode. As one example, to alter the BDDE surface from H-termination to 0-termination, anodic treatment can be performed in an electrochemical cell. At step 1014, the example system applies an electrochemical treatment in a sulfuric acid solution. As one example, the sulfuric acid solution can be charged at +2 V vs. silver/silver chloride, Ag/AgCl, for 5 min, and can include sulfuric acid (H₂SO₄) at a concentration of 0.5 M. The method 1000 then continues to step 1020.

At step 1020, the example system cleans the treated biochemical sensor electrode by cyclic voltammetry. In some implementations, step 1020 includes at least one of steps 1022 and 1024. At step 1022, the example system cleans an oxygen-terminated BDDE with cyclic voltammetry having a range of −0.5 V to +2.8 V. At step 1024, the example system cleans an oxygen-terminated BDDE with cyclic voltammetry having a range of −0.5 V to +1.5 V. The method 1000 then continues to step 1030.

At step 1030, the example system coats the cleaned electrode with an interferent biofouling mitigation solution. In some implementations, step 1030 includes at least one of steps 1032 and 1034. At step 1032, the example system drop casts the interferent biofouling mitigation solution on a BDDE. At step 1034, the example system coats the BDDE with a Nafion solution. Nafion coating can be performed by drop casting 1.8 μL 5 wt % Nafion solution on the biochemical sensor electrode 320. The method 1000 then continues to step 1040.

At step 1040, the example system dries the interferent biofouling mitigation solution to form an interferent biofouling mitigation layer on the electrode. In some implementations, the method 1000 ends at step 1040.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

What is claimed is:

1. A device comprising:
an electrode electrically responsive to presence of a biochemical present within a biofluid; and
one or more biofouling and interferent mitigation layers disposed on the electrode to block transmission of biofouling agents to the electrode and the reaction of interferents on the electrode.

2. The device of claim 1, wherein the electrode comprises a boron-doped diamond electrode.

3. The device of claim 1, wherein the biofouling and interferent mitigation layers comprise an adsorption layer.

4. The device of claim 1, wherein the biofouling and interferent mitigation layers are negatively charged.

5. The device of claim 1, wherein the biofouling and interferent mitigation layers comprise Nafion.

6. The device of claim 1, wherein the electrode comprises at least one of boron-doped diamond, oxygen-terminated boron-doped diamond, and hydrogen-terminated boron-doped diamond.

7. The device of claim 1, wherein the electrode is noninvasively contactable with a biological surface.

8. The device of claim 7, wherein the biological surface comprises at least one of skin and human skin.

9. The device of claim 1, wherein the device comprises a smartwatch.

10. The device of claim 1, further comprising:
a microfluidic layer disposed on the biofouling and interferent mitigation layers and including at least one microfluidic channel.

11. The device of claim 10, wherein the microfluidic channel includes at least one outlet at a first face of microfluidic layer proximate to the electrode, and at least one inlet at a second face of the microfluidic layer opposite to the first face.

12. The device of claim 10, wherein the microfluidic layer comprises at least one flexible layer.

13. A method comprising:
obtaining a biofluid sample;
mitigating an interferent characteristic associated with the biofluid sample;
mitigating a biofouling characteristic associated with the biofluid sample;
obtaining a biochemical characteristic associated with the biofluid sample; and
extracting one or more biochemical signals from a raw electrochemical readout with a baseline estimation algorithm.

14. The method of claim 13, wherein the biofluid comprises at least one of sweat, saliva, human sweat, and human saliva.

15. The method of claim 13, wherein the biofouling and the interferent characteristics are associated with at least one of uric acid, tyrosine, tryptophan, ascorbic acid, histidine, and methionine, a protein, a peptide, a lipid, and an amino acid.

16. The method of claim 13, wherein the biochemical characteristic is associated with acetaminophen.

17. The method of claim 13, further comprising:
contacting an electrode noninvasively with a biological surface,
wherein the obtaining the biofluid sample further comprises obtaining the biofluid sample onto the electrode, and
wherein the obtaining the biochemical characteristic further comprises obtaining the biochemical characteristic associated with the biofluid sample by the electrode.

18. The method of claim 13, further comprising:
contacting an electrode including one or more biofouling and interferent mitigation layers noninvasively with a biological surface,
wherein the mitigating the biofouling characteristic further comprises mitigating the biofouling characteristic associated with the biofluid sample by the biofouling mitigation layer,
wherein the mitigating the interferent characteristic further comprises mitigating the interferent characteristic associated with the biofluid sample by one or more of adjusting surface chemistry of the electrode and incorporating an interferent mitigation layer.

19. The method of claim 18, further comprising:
repelling surface-active agents and electroactive interferent molecules associated with the biofluid sample,
wherein the surface-active agents comprise the biofouling characteristic, and the electroactive interferent can react on the electrode surface and confound the biochemical signal; the biofouling and interferent mitigation layer repel these molecules from approaching the electrode surface via electrostatic force and size-dependent filtering effect.

20. A method of manufacturing an electrode, the electrode being electrically responsive to a biochemical in a biofluid and mitigating a biofouling characteristic associated with an interferent in the biofluid, the method comprising:
applying an anodic treatment to a boron-doped diamond electrode;
coating the electrode with a Nafion solution; and
drying the Nafion solution to form a mitigation layer on the electrode.
